(19) United States
(12) Patent Application Publication
(10) Pub. No.: US 2003/0036071 A1
(43) Pub. Date: Feb. 20, 2003

(54) Computational method for inferring elements of gene regulatory network from temporal patterns of gene expression

(76) Inventor: Alex Lukashin, Charlestown, MA (US)

Correspondence Address: Gary L. Creason, BIOGEN, INC., 14 Cambridge Center, Cambridge, MA, 02142, US

(21) Appl. No.: 10/140,556

(22) Filed: May 7, 2002

Related U.S. Application Data
(63) Continuation of application No. PCT/US00/30814, filed on Nov. 10, 2000.
(60) Provisional application No. 60/165,120, filed on Nov. 12, 1999.

(51) Int. Cl. C12Q 1/68; G06F 19/00; G01N 33/48; G01N 33/50

(52) U.S. Cl. 435/6; 702/20

(57) ABSTRACT

A computational method designed to extract information about gene regulatory network from raw gene expression data sets that are comprised of a time course of expression levels is disclosed. At a first step in this method, genes with similar temporal expression profiles are clustered into modules characterizing by distinct expression signatures. These fundamental patterns of gene expression are analyzed using the assumption that temporal profiles are shaped by interactions between genes belonging to different modules. The underlying genetic connectivity is retrieved using an optimization procedure developed in computational neurobiology for extracting information about neural circuitry. The objective is to find an optimal regulatory structure making calculated temporal patterns as close as possible to experimental data. A set of algorithms was used to evaluate statistical significance of putative regulatory connections derived from gene expression patterns. The method was utilized to identify regulatory subnetworks underlying the response of yeast cells to treatment with acid and alkaline conditions. Expression profiles of about 1600 genes that showed a significant change in expression during a time course were analyzed according to the method of the invention. The genes were clustered into 39 distinct modules and statistically significant connections between 16 modules representing most variable genes were identified and mapped to a sub-network of known connections. The results demonstrate that the computational method may be a useful tool both in elucidating of crucial elements of genetic network structure and in predicting novel regulatory connections based on gene expression.

### RELATED APPLICATIONS

This is a continuation of PCT/US00/30814, filed on Nov. 10, 2000, which claims priority from U.S. provisional application Serial No. 60/165,120 filed on Nov. 12, 1999.

### BACKGROUND

Genetic methods are useful for the determination of gene function and the interactions between genes and gene products. Genetic methods, however, are laborious and can provide information on a limited number of genes at any one time. The development of computer-based computational tools are providing the means by which genetic data can be stored, sorted, grouped and rapidly analyzed using a variety of algorithms. In genome projects, such tools allow the storage of large amounts of gene sequence information and the rapid analysis of the sequence information to map the gene sequences to their locations on chromosome and to predict protein sequence, structure and function from the sequence data.

Computer-based computational tools are being developed and applied to the study of organism's genomes to determined the sequence and placement of its genes and their relationship to other sequences and genes within the genome or to genes in other organisms. The relationships between genes both within an organism and between organisms is of significant interest in biomedical and pharmaceutical research, for instance to identify genes that may be suitable targets for drug development and to assist in the evaluation of drug efficacy and resistance.

### SUMMARY OF THE INVENTION

The present invention provides a method of estimating and displaying the level of interaction (or “strength of connection”) between a plurality of gene clusters. The method involves providing a database including a plurality of gene clusters, preferably the database includes a plurality of gene expression profiles together with biological annotations detailing the source and any interpretation of the expression profile information. The method further involves selecting a set of gene clusters and estimating the level of interaction between each gene cluster in the set using computer assisted optimization of a connectivity matrix.

The invention provides a computer program product comprising a computer-useable medium having computer-readable program code embodied thereon relating to a database including multiple expression profiles. The computer program includes computer-readable program code for selecting a set of gene clusters, and estimating and displaying the level of interaction between gene clusters in the selected set.

The method of the invention may be used for the analysis of expression profiles from both prokaryotic and eukaryotic cells. Use of the method of the invention is exemplified using yeast cells with which the expression profiles of about 1600 genes were measured under both alkaline and acidic conditions.

The following terms are used through the specification. Definitions of these terms are provided to assist in understanding the specification, but do not necessarily limit the scope of the invention.

An “expression profile” means the level of expression of a gene, observed as the number of mRNA molecules transcribed from a given gene, that is measured at one or more time points during cellular differentiation or cellular response to stimuli.

A “gene cluster” or “module” means genes that have been grouped together on the basis of their having similar expression profiles during cellular differentiation or cellular response to stimuli. The gene cluster is assigned an expression profile which is the averaged expression profile of the clustered genes.

The “level of interaction” or “strength of connection” means the computed level of interaction between one gene cluster and its proposed target gene cluster, which connection can be positive (activation of the target gene cluster), negative (inhibition of the target gene cluster) or equal to zero (no connection between the selected gene cluster and its proposed target gene cluster).

“Connectivity matrix” means a matrix of coefficients in which each coefficient represents the strength of connection between two gene clusters.

Throughout the text of the specification published articles will be referred to by reference number and the list of the published articles can be found on the final page before the claims.

### BRIEF DESCRIPTION OF THE DRAWINGS

FIG. 1. Comparison between experimentally measured and calculated temporal expression profiles. Each template shows the data for one of 16 “variable” clusters (see Materials and Methods) in both acid (the left part of template) and alkaline (the right part) conditions, which are separated by a vertical line. Inside each template “C” stands for “cluster number”. The numbering of clusters corresponds to their numbers in the whole set of 39 clusters (see the web site http://www.wi.mit.edu/young/). The ordinate is the expression level scaled from 0 to 1. The abscissa is the time-axis: the left half of the axis is 0-100 minutes interval for acid condition, whereas the right half is the same time interval for alkaline condition. Filled circles: experimental expression data represented by centroids (average patterns for genes in the clusters). Each pattern includes 14 time points: 0, 10, 20, 40, 60, 80 and 100 minutes in acid condition followed by 0, 10, 20, 40, 60, 80 and 100 minutes in alkaline condition. The diameter of circles is approximately equal to a half of typical standard deviation for patterns in a cluster. Solid curves: temporal expression patterns calculated by means of Eq.1,2. The profiles for acid and alkaline conditions were obtained using the averaged connectivity matrices \overline{R}ᵢₖ^{acid} and \overline{R}ᵢₖ^{alkaline} derived as described in the text. The deviation of calculated profiles from experimental data (Eq. 3) varies from 0.023 (cluster # 5, alkaline condition) to 0. 145 (cluster # 41, acid condition) with average values 0.074 and 0.055 for acid and alkaline conditions, correspondingly.

FIG. 2. Schematic representation of connectivity matrices \overline{R}ᵢₖ^{acid} and \overline{R}ᵢₖ^{alkaline} acid and alkaline conditions, correspondingly) for 16 “variable” modules. The module numbers are shown in rows and columns next to each matrix. The signs “+” and “−” mark the elements that are significant and positive or negative; the sign “.” marks insignificant elements. The entry \overline{R}ᵢₖ lies at the intersection of the i-th row and the k-th column; the direction of connection is from k to i (i<−k ). For instance, module # 24 (column) activates module # 4 (row), whereas module # 4 (column) inhibits module # 16 (row). A and C—connectivity matrices derived in the model of the 16 interacting modules (the 16×16 model). These matrices were used to calculate temporal profiles shown in FIG. I by solid curves. B and D—connectivity matrices for the same 16 modules derived in the model in which interactions between all 39 modules were allowed (the 39×39 model); these matrices represent 16×16 sub-matrices of larger 39×39 matrices. Highlighting is used to compare matrices derived in different models: yellow—the connection is significant in matrix A and insignificant in matrix B or vice versa (the same for the pair C and D); pink/blue—the connection is significant and positive/negative in both A and B (C and D) matrices; green—the connection is significant and positive in matrix A but negative in matrix B or vice versa (the same for the pair C and D).

FIG. 3. Invariant connectivity matrices derived from expression profiles measured in acid and alkaline conditions. The positive and negative connections, marked by signs “+” and are invariant with respect to the model used (16×16 or 39×39). A—the acid matrix derived from matrices A and B in FIG. 2; B—the alkaline matrix derived from matrices C and D in FIG. 2. Highlighting is used to compare the acid and alkaline matrices: yellow—the connection is significant in matrix A and insignificant in matrix B or vice versa; pink/blue—the connection is significant and positive/negative in both A and B matrices; green—the connection is significant and positive in matrix A but negative in matrix B or vice versa.

### DESCRIPTION OF THE PREFERRED EMBODIMENTS

The rapid advance of microarray technologies to monitor simultaneously expression profiles of thousands of genes has stimulated the development of computational tools to organize efficiently such data in system-level conceptual schemes (1-13). Particularly, various algorithms for clustering temporal expression patterns measured during cellular differentiation or response (2,7,10,12) have clearly proven valuable for exploration of gene regulatory networks. The purpose of the cluster analysis is to group together genes with similar expression profiles and, on the basis of the resulting partition, to assess potential similarity of the genes' function. A natural next question is what is beyond the clustering? In other words, given a set of clusters having characteristic shapes of expression profiles, how to extract information about interconnectivity and mutual regulation of genes belonging to different clusters. In general, shapes of gene expression profiles can be interpreted in a manner that specific pathways independently regulate specific genes (or clusters of genes), and therefore changes in expression observed for the distinct clusters are not related to each other. A more realistic concept is that the pathways are heavily interconnected so that the shapes of expression profiles convey information about underlying regulatory network. As a straightforward example, one may expect that a change in expression level of a transcriptional factor should affect expression of its target gene. In a broad sense, the interplay between different expression patterns can reflect connectivity through cis and trans elements, protein-protein and protein-signaling factor interactions (2), as well as a “crosstalk” between signaling pathways (14). This invention provides a computational scheme for recognition of those elements of presumed regulatory network that are crucial for the shaping of distinct temporal expression profiles. The method of the invention essentially implements aphenomenological model of gene regulation that is specifically constructed to interpret temporal expression profiles. First of all, genes with similar patterns of expression are clustered using published computational tools referred to above. The set of the genes fallen into the same cluster will be called the “module” to emphasize that these genes are indistinguishable in the framework of the method. Each distinct module of genes is characterized by its unique expression “signature”. These modules are the basic operational units in the method. Second, we assume that a module can receive input from all other modules and change the level of expression responding to the integrated signal. We do not specify biological mechanisms underlying the input, integration of inputs and response. The signal from a module is just the product of the module's expression level times the strength of connection between the module and its target. Connection can be positive (activation), negative (inhibition) or equal to zero (no connection). Therefore, given a set of connections between modules (the connectivity matrix), the temporal expression profiles are interrelated so that each individual profile emerges as the result of communications between all modules within the ensemble. Third, since the calculated expression profiles are sensitive to the structure of connectivity, our objective is to solve the inverse problem: namely, given a set of experimentally measured expression patterns, we aim to find the connectivity matrix (or subset of matrices, in case of redundancy) that would create the temporal profiles whose shapes are as close as possible to experimental data. The resulting connectivity between distinct modules of genes can be interpreted as a putative regulatory network. The method of the invention described above was applied to identify elements of gene regulatory network underlying the response of yeast cells to treatment with acid and alkaline conditions. The whole-genome mRNA abundance was measured in both conditions at 7 time points across 100 minutes interval. About 1600 genes that showed significant changes in expression and the distinct expression profiles were clustered and the gene clusters, or modules, were used to estimate the connectivity between modules of genes. The application of the method of the invention to shuffled expression profiles provided a measure of significance of the resulting connectivity matrix. Since the method of the invention did not utilize any a priori knowledge of gene regulation in yeast the method was validated by a mapping of predicted connections to a sub-network of expected interactions “transcriptional factor—target gene”. The estimated strength of connections between the modules determined through application of the method of the invention also provides a basis for recognition of novel elements of the regulatory network that are interesting for further exploration.

The method of the invention is based on a close mathematical analogy between the problem of identifying gene regulatory networks, using temporal expression profiles, and the problem of identifying network of synaptic connections in neural systems, using temporal profiles of neurons' firing rates. For the latter problem, computational tools are well elaborated and widely used in studies of cortical circuits (e.g., refs. 15-17). Below, we outline the basic equations applied to gene regulatory networks drawing a parallel with neural networks.

Model.

Consider an ensemble of N units (N modules of genes or N model neurons), each one characterized by a time-dependent variable Vᵢ(t) that represents activity (level of gene expression or firing rate for neural systems) of the i-th unit (i=I,-, N) at the instant of time t. The activities are normalized so that values Vᵢ(t) vary within the interval between 0 and 1. Each unit receives an integrated input Uᵢ(t) from all other units via a set of connections (gene regulatory connections or synaptic connections). The signal that a particular unit number k sends to the unit number i is the product of the k-th unit activity Vₖ(t) times the connection strength Rᵢₖ, which can be positive (activation), negative (inhibition) or equal to zero (no connections). Connections Rᵢₖ are directed (i<−k ) and may not be symmetric (R_{ik≠}Rₖᵢ). Conventionally, the integrated input Uᵢ(t) is assumed to change in time according to the “circuit” equation (18,19):

$${U}_{i} ( t ) + τ \frac{ⅆ {U}_{i} ( t )}{ⅆ t} = {\sum }_{k = 1}^{N} {R}_{ik} {V}_{k} ( t ) \qquad [ 1 ]$$

where τ is a characteristic time constant that regulates how fast a unit accumulates the overall input signal defined by the right-hand side of Eq. 1. The larger is the value of τ, the longer time is required to accumulate the signal. Each unit transforms the input Uᵢ(t) to the output activity Vᵢ(t) acting as a nonlinear amplifier, which saturates when the input exceeds a threshold value. The detailed form of this transformation does not affect the function of the ensemble (15-19). We take the simplest form:

$${V}_{i} ( t ) = \{ \begin{matrix}1 & if {AU}_{i} ( t ) + {S}_{i} > 1 , \\ {AU}_{i} ( t ) + {S}_{i} & if 0 \leq {AU}_{i} ( t ) + {S}_{i} \leq 1 , \\ 0 & if 0 > {AU}_{i} ( t ) + {S}_{i} ,\end{matrix} \qquad [ 2 ]$$

where A is the gain of the unit in the linear operating region, Sᵢ represents a spontaneous level of expression that would be observed if the unit is not affected externally (spontaneous firing rate of a neuron). Equations 1 and 2, taken for all units, constitute the system of nonlinear equations that governs the temporal behavior of the ensemble.

Optimization Scheme.

In the framework of the model, the connectivity matrix Rᵢₖ essentially determines the shapes of all temporal expression profile Vᵢ(t). We aim to solve the inverse problem, that is, to identify the structure of connectivity matrix that would make the difference between the calculated and measured expression profiles as small as possible. Let the experimentally obtained levels of expression Wᵢ (i=1, . . . ,N) be given at M time points t=t₁, . . . ,t_{M}. As a measure of the difference between the “desired” expression patterns, Wᵢ(t), and the calculated temporal profiles, Vᵢ(t), we take the root mean square deviation

$$E = {( \frac{1}{NM} {\sum }_{i = 1}^{N} {\sum }_{m = 1}^{M} {[ {W}_{i} ( {t}_{m} ) - {V}_{i} ( {t}_{m} ) ]}^{2} )}^{1 / 2} \qquad [ 3 ]$$

which is a function of N² adjustable parameters Rᵢₖ (i, k=1, . . .,N). To minimize the E value we apply the simulated annealing algorithm (20). At each iterative step, the set of connection strengths Rᵢₖ is changed in a random manner (Rᵢₖ^{p;→}Rᵢₖ^{new}); the specific way of this change has no effect on the procedure (ref. 21; see Materials and Methods for details). The new root mean square deviation E^{new} is calculated (Eqs. 1,2 and 3) and compared with the previous value E^{old} is larger than E_{new}, the new set of parameters Rᵢₖ^{new} is unconditionally accepted and used as the starting point for the next iteration. Otherwise, the new set Rᵢₖ^{new} is accepted with probability exp[−(E^{new}−E^{old})/T], where the parameter T can be interpreted as the “temperature”, if the E value is treated as the “energy” of the system. This algorithm guarantees that after a sufficient number of iterative steps the system obeys the Boltzmann distribution at a given temperature. Consequently, if the temperature tends to zero slowly enough, the system reaches the global minimum of the root mean square deviation (Eq. 3). Routinely, we used exponential cooling schedule Tₙ₊₁=cTₙ, where ₙ is the step number and the value 1−c is positive and close to zero. Note, although the simulated annealing algorithm guarantees to eventually find the optimal solution, it cannot guarantee that the optimal value of E will be E=0.

Thus, the algorithm described above makes it possible to identify the optimal regulatory network in terms of the optimal connectivity matrix Rᵢₖ. However, while solving the inverse problem, one may expect a redundancy of solution: a large number of different connectivity matrices Rᵢₖ can result in the same optimal value of E. This issue will be addressed in the section Results and Discussion.

### Materials and Methods

Clustering and Normalization.

The whole-genome experimental data provided us with 7 time points (including the zero time point) in both acid and alkaline conditions across 100 minutes for each type of stimulation. We analyzed 1618 genes that showed more than 3-fold change in transcriptional level in either of the two conditions. The variance-normalized expression patterns for each of these 1618 genes were concatenated so that the zero time point for the alkaline condition followed the last time point (100 minutes) for the acid condition. The concatenated profiles were clustered into 39 clusters of 10-80 genes per cluster, using the Self-Organizing Map algorithm (10). The concatenation made it possible to group together genes whose temporal behavior was similar in both acid and alkaline conditions. Within each cluster, the expression profile represented by the average pattern for genes in the cluster was normalized to have the minimum and maximum levels of expression equal to 0 and 1, correspondingly. This normalization set up the same scale for the measured and calculated expression patterns and eased the comparison of their shapes. Of these 39 clusters, we selected 16 “variable” clusters (726 genes) for which the difference between the minimum and maximum levels of expression was greater than or equal to 0.5 in acid condition, and the same was in alkaline condition. The raw gene expression data, graphical representation of all clusters along with the distribution of genes over the clusters are available at the web site http://www.wi.mit.edu/young/.

Computational Issues.

Given a current connectivity matrix Rᵢₖ the system of coupled non-linear equations (Eqs. 1,2) was solved as the initial value problem, Uᵢ(0)=0, using a forth-order Runge-Kutta formula with automatic control of the step size during the integration. The spontaneous levels of expression (Sᵢ in Eq. 2) were set: Sᵢ=Wᵢ (0), so that if the units were disconnected (all connection strengths Rᵢₖ=0) the expression would be at the steady level equal to the expression level measured at the zero time: Vᵢ(t)=Wᵢ (0). Other parameters: the characteristic time constant τ (Eq. 1) was chosen: τ=10 minutes; the gain parameter (Eq. 2) A=0.5. For each minimization trial, the connection strengths Rᵢₖ were initialized to uniform random values between −1 and 1. During the annealing procedure, new probe values Rᵢₖ were selected randomly from the same interval [−1,1] without assuming symmetry. One step included a change of one parameter chosen at random and the entire recalculation of all expression patterns. The temperature at the initial stages of the simulated annealing was chosen to have accepted practically all states of the system. The cooling parameter 1−c was varied within the interval from 10⁻⁷ to 10⁻⁵ depending on the rate of convergence.

### Results and Discussion

Redundancy and Self-Averaging.

As expected, a direct approach to identify gene regulatory network by minimizing the deviation of calculated expression profiles from experimental data (Eq. 3) ran into a redundancy problem: a very large number of different connectivity matrices R^{ik} offered sub-optimal solutions. Therefore, in the context of the present work, a fundamental question is how to recognize the most crucial non-random connections. We have found that a simple averaging procedure can cope with the redundancy problem as follows. For the set of 16 interacting modules of genes representing 16 “variable” clusters (see Materials and Methods), the minimization procedure as described above was repeated K times and K distinct sub-optimal matrices 16×16 were averaged by calculating the mean value of each matrix element and the standard deviation \overline{R}ᵢₖ. This was done separately for acid and alkaline conditions. Routinely, the minimization procedure ended up with the E value (Eq. 3) ranging within the interval 0.061±0.003, for acid condition, and 0.044±0.003, for alkaline condition. Obviously, if the sub-optimal matrices were quasi-random all elements of averaged matrix \overline{R}ᵢₖ would tend to zero as ±1/\sqrt{K}. In contrast, when the number of trials K approached 102, about a half of matrix elements \overline{R}ᵢₖ stabilized around the values that were significantly above the random noise level:

$${\vert {\overline{R}}_{ik} \vert}_{- 2 {D}_{ik} > 1} \sqrt{K}$$

(data not shown).

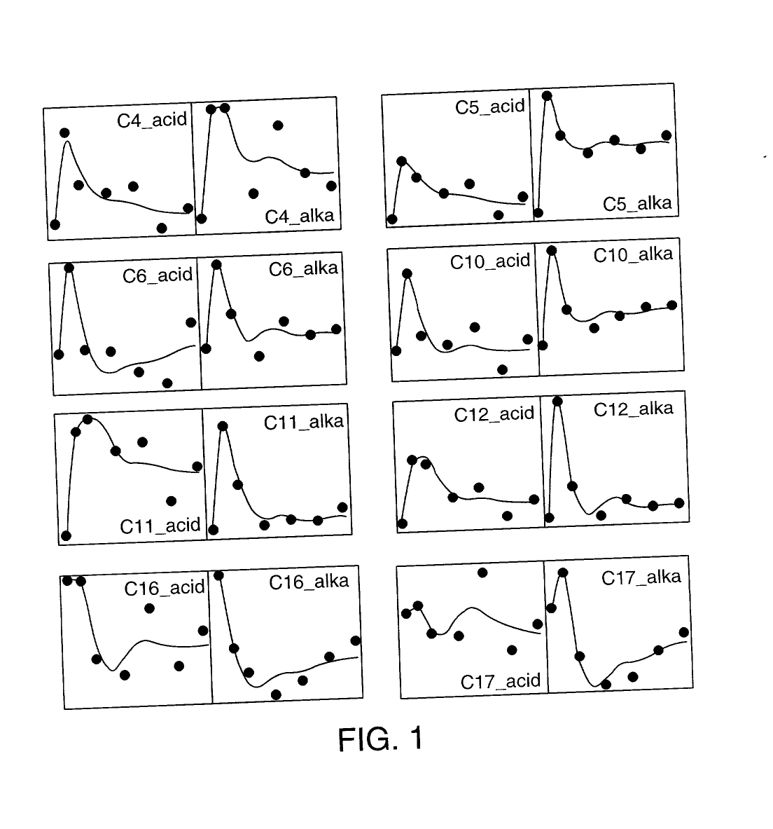

Although the averaging procedure exposed the stable non-random connections it was not clear a priori whether our model could reproduce experimental expression profiles if the averaged connection strengths \overline{R}ᵢₖ were used in Eqs. 1, 2. In other words, whether the model belongs to the class of so-called self-averaging systems, for which the output (in our case, temporal expression profiles) averaged over different inputs (connectivity matrices) is equal or close to the output calculated for averaged input. The temporal expression profiles calculated using connectivity matrices \overline{R}ᵢₖ^{acid} and \overline{R}ᵢₖ^{alkaline}, each derived by averaging over 100 minimization trials, are shown in FIG. 1 (solid curves) along with normalized experimental profiles (filled circles). Quantitatively, if the profiles presented in FIG. 1 are compared, the root mean square deviation E (Eq. 3) is equal to 0.074 for acid condition and 0.055 for alkaline condition. Another pair of 100 minimization trials may result in slightly different averaged matrices and different E value. Summarizing, we found that the deviation E for averaged matrices ranged within the interval E=0.072±0.005 for acid condition and E=0.053±0.004 for alkaline condition. A further increase of the number of trials K did not change the conclusion. The deviation of calculated expression profiles from experimental data obtained for averaged matrices is slightly greater than the deviation that can be reached in each individual minimization trial (0.07 vs. 0.06 and 0.05 vs. 0.04). However, the absolute values of deviation 0.05-0.07 are still relatively small (e.g., at initial stages of the minimization procedure, the deviations are of the order of 0.5-0.6), and the calculated and experimental expression profiles are in a reasonable agreement (FIG. 1). In addition, the shapes of temporal profiles are weakly affected by a variation of connection strengths around the average values \overline{R}ik. We calculated the expression profiles using “noisy” connectivity matrices defined as Rik=\overline{R}ᵢₖ+2αDik, where α was a random number from the interval [−1, 1]. The maximum values of deviation E (Eq. 3) recorded in a session of 1000 trials with randomly modified matrices were 0.079 and 0.062 for acid and alkaline conditions, correspondingly. These results demonstrate feasibility of the averaging procedure for extracting stable connections between interacting modules of genes.

Robustness of Connections.

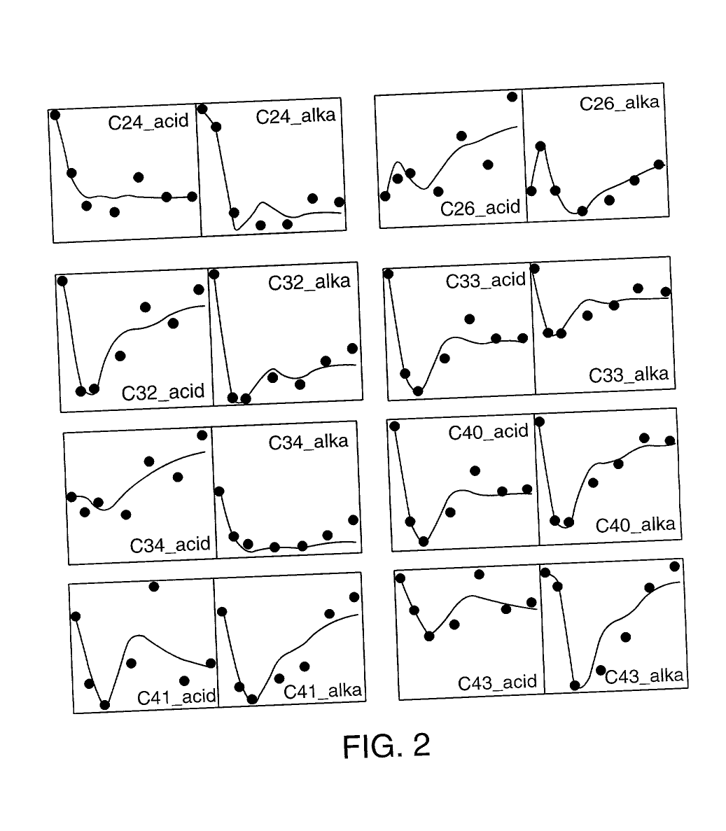

The elements constituting an averaged connectivity matrix \overline{R}ᵢₖ can be conventionally divided into two groups, “significant” or “insignificant”, judging from whether or not the absolute value of \overline{R}ᵢₖ is above or below a level of random noise. To make the criterion of significance more stringent, we applied the same optimization scheme to shuffled experimental data. Specifically, in each minimization trial, we randomly shuffled time positions of expression levels within each profile, leaving their absolute values unchanged. The acid and alkaline profiles were not mixed. Since the shuffling is a more conservative procedure than a complete randomization, the resulting averaged matrix \overline{F}ₘₙ provides a more reliable reference than the average taken over random matrices. We assigned a matrix element \overline{R}ᵢₖ to the class of “significant” connections, if it satisfied the requirement

$${\vert {\overline{R}}_{ik} \vert}_{- 2 {D}_{ik} > max} \vert {\overline{F}}_{mn} \vert ,$$

where maximum was taken over all entries. So far we reported the results obtained for the model in which 16 “variable” modules were involved in interactions (the 16×16 model). To further test the reliability of the results, we repeated the whole optimization procedure using an extended model in which all 39 modules were allowed to interact with each other (the 39×39 model). The sub-matrix 16×16 for “variable” modules can be extracted from the matrix 39×39 to compare outputs of the two models. This comparison provides a valuable test on the robustness of solution: if a connection is identified as significant in the 16×16 model, it should remain significant in the extended 39×39 model as well, even though 23 new “players” are added in the ensemble of interacting modules: Four connectivity matrices derived in both acid and alkaline conditions using both the 16×16 and 39×39 models are depicted in FIG. 2. The significant matrix elements are marked by the signs ‘+’ and “−” for positive and negative connections. The elements highlighted pink (positives) and blue (negatives) represent model-invariant significant connections. When matrices A and B (different models for acid condition) are compared, it is apparent that the number of similar connections significantly exceeds the number expected by chance: 31 positives vs. 9 expected and 39 negatives vs. 13 expected. For alkaline condition (compare matrices C and D), we have: 37 positives vs. 9 expected and 42 negatives vs. 12 expected. The number of expected coincidences was estimated assuming a random distribution of a given number of significant elements within a matrix 16×16. Remarkably, there is only one case in which a connection has opposite signs in different models (the connection between module # 10 and module # 41, acid condition), whereas the expected number of such cases is equal to 21 and 20 in acid and alkaline conditions, correspondingly. These results show that a majority of significant connections is invariant with respect to the model from which the connectivity was derived.

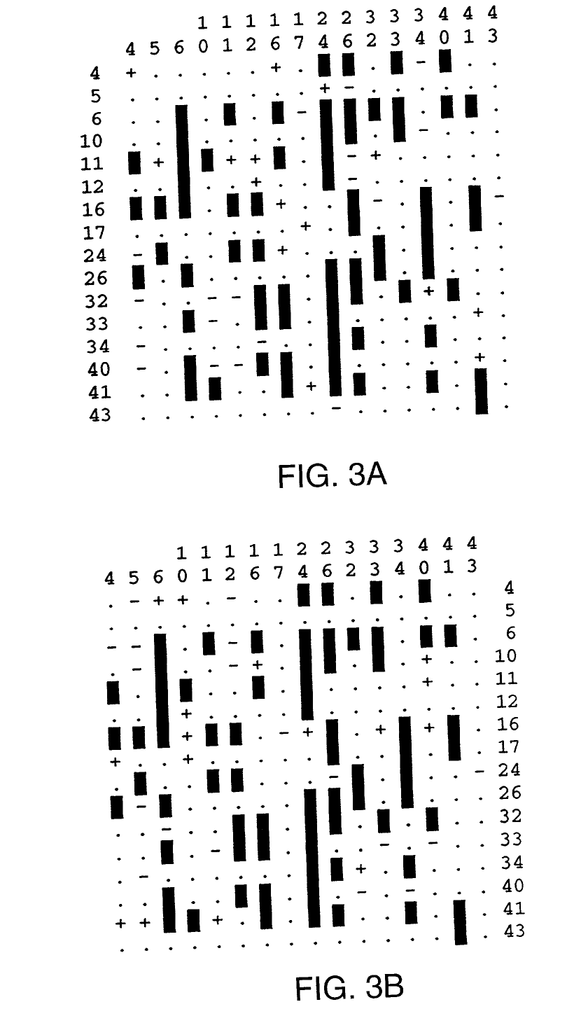

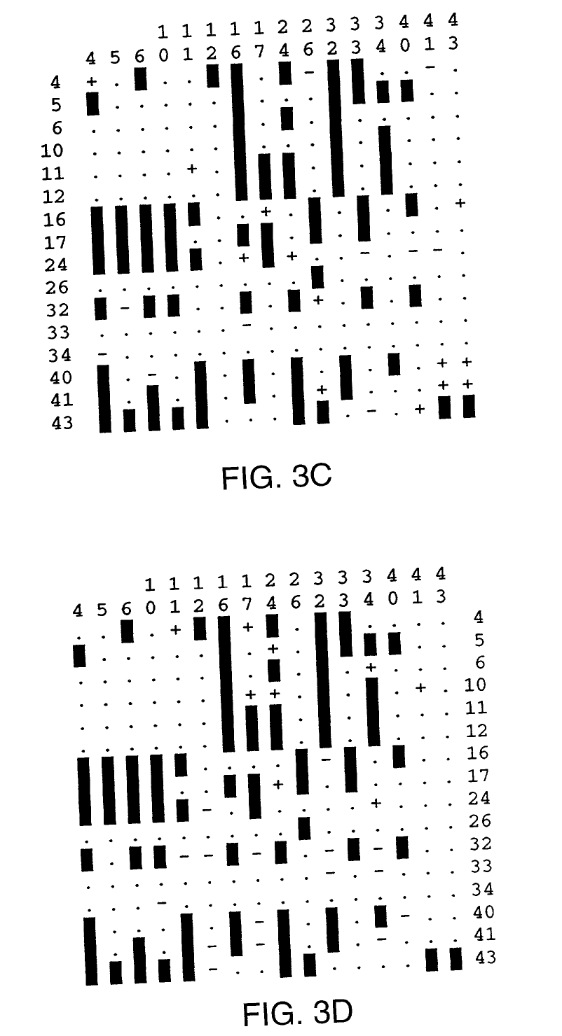

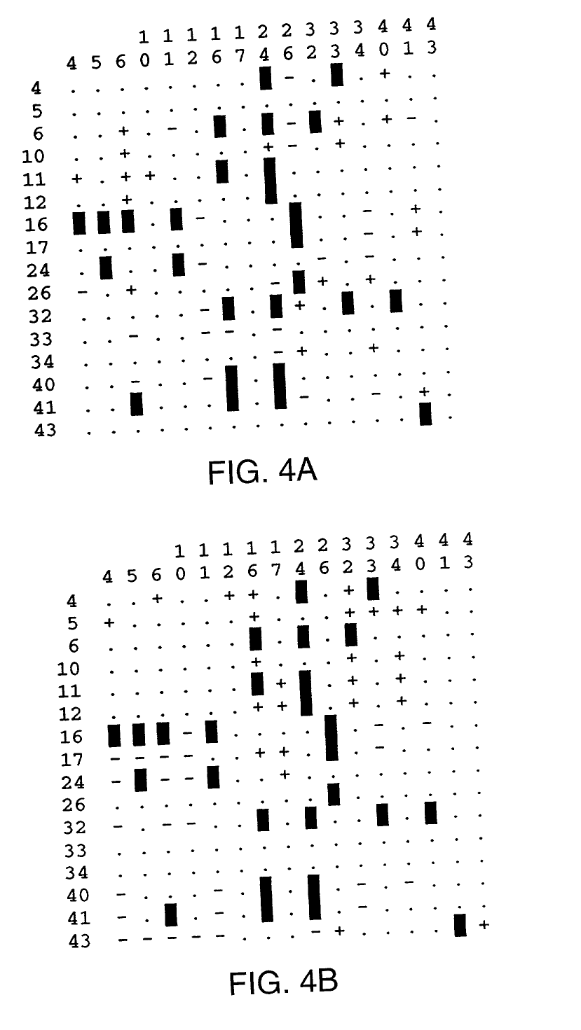

To visualize the similarity and difference between connectivity matrices derived from expression profiles measured in acid and alkaline conditions, we placed in FIG. 3 the acid matrix (A) and alkaline matrix (B) where only the model-invariant elements are left illuminated. If matrices A and B in FIG. 3 are compared, the number of similar positive and negative elements also exceeds the number of coincidences expected by chance: 10 positives vs. 4 expected and 14 negatives vs. 6 expected. Only 3 connections have opposite signs in different matrices (11 expected). In fact, the similarity between matrices A and B (FIG. 3) is not surprising, given a partial similarity between expression profiles measured in different conditions (see FIG. 1).

Predictions and Comparison.

The connections highlighted in FIG. 3 summarize the outcome of our modeling. They represent elements of a putative regulatory network underlying the shapes of temporal expression profiles observed in acid and alkaline conditions. Yellow color illuminates connections that are unique with respect to different treatments. Patterns of these connections seen in FIGS. 3A and B can be interpreted as gene regulatory sub-networks involved in response to different stimulation. Pink and blue highlighting emphasizes those positive and negative connections that remain stable regardless of the type of treatment used. These connections are likely the most crucial for gene regulation in yeast.

We stress that our method predicts connections between modules (clusters) of genes, and individual genes belonging to the same cluster are indistinguishable from each other. In spite of this uncertainty, we attempted to compare the connectivity predicted in the framework of our model with regulatory connections documented on the basis of experimental data. Among genes constituting 16 “variable” clusters, there are 4 genes whose products are known as transcriptional regulators: XBP1, RME1, ABF1 and BAS1. They belong to clusters # 5, 11, 17 and 33, correspondingly. The target clusters and type of connectivity predicted for these 4 regulators are listed in Table 1, along with available information about the genes that are known as targets for the 4 regulators. For instance, regulator XBP1 is known as a repressor. This gene falls into module # 5 predicted as a repressor for modules # 16 and 24 in acid condition (FIG. 3A) and, additionally, for modules # 17 and 43 in alkaline condition (FIG. 3B). Consistently, Table 1 shows that cluster # 43 includes gene VAP1 known as a target for regulator XBP1. An interesting example demonstrates module # 17. According to prediction (FIG. 3B), it activates itself. Indeed, both genes ABF1 and YPT10 known as a pair activator—target belong to this cluster (Table 1). On the other hand, module # 17 is a predicted target for module # 33 (FIG. 3B). This is also consistent with available information (Table 1) that the product of gene BAS I from cluster # 33 regulates expression of gene PH05 from cluster # 17. Of course, the matches between predicted and expected connections presented in Table 1 may serve only as a positive control and do not assert the validity of the method in general. However, it should be noted that putative regulatory connections have been derived on the basis of only one assumption that the shapes of expression profiles emerge as a result of interactions between modules of genes, and no specific knowledge about gene regulation in yeast has been used. The outcome of our analysis exemplified in FIG. 3 and Table 1 suggests novel regulatory connections identified directly from raw expression array data sets. This information can be used for further exploration of interactions between specific genes belonging to a distinct clusters, as well as for annotation of new candidates whose function is unknown. In conclusion, we believe that our method provides a useful tool to construct a skeleton of gene regulatory network on which more detailed biological information might be overlaid.

TABLE 1
Mapping of predicted connections to a
sub-network of expected interactions

| Predicted | | Expected |
|---|---|---|
| Target | Type of connection | Gene |

| Regulator | cluster | Acid | Alkaline | known as target |
|---|---|---|---|---|
| XBP1 | 16 | R | R | |
| cluster #5; | 17 | | R | |
| known as | 24 | R | R | |
| repressor | 43 | | R | VAP1 |
| RME1 | 6 | R | | |
| cluster #11; | 16 | R | R | |
| known as | 24 | R | R | |
| repressor | 40 | | R | CLN2 |
| | 41 | | R | |
| | 43 | | R | |
| ABF1 | 11 | | A | |
| cluster #17; | 12 | | A | MSS51 |
| known as | 17 | | A | YPT10 |
| activator | 24 | | A | |
| | 4 | A | A | |
| BAS1 | 5 | | A | |
| cluster #33 | 6 | A | | |
| | 10 | A | | |
| | 16 | | R | |
| | 17 | | R | PH05 |
| | 32 | R | R | |

The first column shows name of known regulator, number of cluster where the gene is from, and a description (repressor or activator, if known). Next three columns present predicted target cluster numbers and type of connection between the regulator and targets. These data are taken from FIG. 3: “A” stands for positive regulation (activator), “R” stands for negative regulation (repressor). The rightmost column gives available information about the genes that are know as targets for the 4 regulators and fallen into one of 16 “variable” clusters. The names of these target genes are shown in the rows corresponding to clusters where they are from.

1. DeRisi, J. L., Iyer, V. R. & Brown, P. O. (1997) Science 278, 680-686.

2. Wen, X., Fuhrman, S., Michaels, G. S., Carr, D. B., Smith, S., Barker, J. L. & Somogyi, R. (1998) Proc. Natl. Acad. Sci. USA 95, 334-339.

3. Roth, F. P., Hughes, J. D., Estep, P. W. & Church, G. M. (1998) Nature Biotechnol. 16, 939-945.

4. Khan, J., Simon, R., Bittner, M., Chen, Y., Leighton, S. B., Phoida, T., Smith, P. D., Jiang, Y., Gooden, G. C., Trent, J. M. & Meltzer, P. S. (1998), Cancer Res. 15, 5009-5013.

5. Cho, R. J., Campbell, M. J., Winzeler, E. A., Steinmetz, L., Conway, A., Wodicka, L., Wolfsberg, T. G., Gabrielian, A. E., Landsman, D., Lockhart, D. J. & Davis, R. W. (1998) Mol. Cell 2, 65-73.

6. Holstege, F. C. P., Jennings, E. G., Wyrick, J. J., Lee, T., Hengartner, C. J., Green, M. R., Golub, T. R., Lander, E. S. and Young, R. A. (1998) Cell 95, 717-728.

7. Eisen, M. B., Spellman, P. T., Brown, P. O. & Botstein, D. (1998) Proc. Natl. Acad. Sci USA 95, 14863-14868.

8. Spellman, P. T., Sherlock, G., Zhang, M. Q., Iyer, V. R., Anders, K., Eisen, M. B., Brown, P. O., Botstein, D. & Futcher, B. (1998) Mol. Biol. Cell 9, 3273-3297.

9. Iyer, V. R., Eisen, M. B., Ross, D. T., Schuler, G., Moore, T., Lee, J. C. F., Trent, J. M., Staudt, L. M., Hudson, J., Jr., Boguski, M. S., Lashkari, D., Shalon, D., Botstein, D., & Brown, P.O. (1999) Science 283, 83-87.

10. Tamayo, P., Slonim, D., Mesirov, J., Zhu, Q., Kitareewan, S., Dmitrovsky, E., Lander, E. and Golub, T. R. (1999) Proc. Natl. Acad. Sci. USA 96, 2907-2912.

11. Alon, U., Barkai, N., Notterman, D. A., Gish, K., Ybarra, S., Mack, D. and Levine, A. J. (1999) Proc. Natl. Acad. Sci USA 96, 6745-6750.

12. Tavazoie. S., Hughes, J. D. Campbell, M. J., Cho, R. J. and Church, G. M. (1999) Systematic determination of genetic network architecture. Nature Genetics 22, 281-285.

13. Perou, C. M., Jeffrey, S. S., van de Rijn, M., Rees, C. A., Eisen, M. B., Ross, D. T., Pergamenschikov, A., Williams, C. F., Zhu, S. X., Lee, J. C., Kashkari, D., Shalon, D., Brown. P. O., & Botstein, D. (1999) Proc. Natl. Acad. Sci. USA 96, 9212-9217.

14. Fambrough, D., McClure, K., Kazlauskas, A. & Lander, E. S. (1999) Cell 97, 727-741.

15. Abbott, L. F. (1994) Q. Rev. Biophys. 27, 291-331.

16. Arbib, M. A. (Ed.) The Handbook of Brain Theory and Neural Networks, (Cambridge, Mass.: MIT Press, 1995).

17. Lukashin, A. V. (1996) Curr. Opin. Neurobioal. 6, 765-772.

18. Hopfield, J. J. (1984) Proc. Natl. Acad. Sci. USA 81, 3088-3092.

19. Kleinfeld, D. (1986) Proc. Natl. Acad. Sci. USA 83, 9469-9473.

20. Kirkpatrick. S., Gelatt, C.D. and Vecchi, M. P. (1983) Science 220, 671-680.

21. Aart, E. H. L. and van Laarhoven, P. J. M. (1987) Simulated annealing: a review of the theory and applications (Kluwer-Academic Publisher, Dordercht).

We claim:

1. A method of estimating interactions between a plurality of gene modules, each one of the gene modules being characterized by a corresponding expression profile representative of an expression level of that one gene module during a time interval, the method comprising:
(A) measuring the expression profile for each one of the gene modules;
(B) predicting the expression profile of each one of the gene modules according to a function of the expression profiles of all the other gene modules and a plurality of coefficients, each of the coefficients representing an amount of effect that the expression profile of one of the modules may have on the expression profile of another one of the modules;
(C) selecting values for the coefficients that minimize a measure of the difference between the measured expression profiles and the predicted expression profiles.

2. A method according to claim 1, further comprising identifying the gene modules from a multiplicity of genes, each one of the genes being characterized by an expression profile representative of an expression level of that one gene during a time interval, identifying the gene modules comprising:
(A) measuring the expression profiles of the multiplicity of genes in an eukaryotic cell; and
(B) clustering genes characterized by similar expression profiles together into one of the modules.

3. A method according to claim 1, wherein selecting values for the coefficients comprises:
(A) assigning initial values to each of the coefficients;
(B) using the coefficients to calculate predicted expression profiles for at least some of the modules;
(C) selecting new values for the coefficients according to a function of a difference between the predicted expression profiles and the measured expression profiles.

4. A method according to claim 1, wherein selecting values for the coefficients comprises using simulated annealing.

5. A method according to claim 1, wherein selecting the values for the coefficients comprises using a mathematical optimization algorithm.

6. A method according to claim 1, wherein selecting values for the coefficients comprises identifying two or more candidates for at least one of the coefficient values and setting the one coefficient value equal to an average of the candidates.

7. A method of estimating interactions between a plurality of gene modules, each one of the gene modules being characterized by an expression level, the method comprising:
(A) measuring the expression level of each one of the gene modules at a plurality of times within a time interval;
(B) calculating predicted values of the expression levels of each one of the gene modules for a plurality of times within the time interval, the predicted value of the expression level of one of the gene modules at a particular time being calculated according to a function of a plurality of coefficients and the predicted or measured values of the expression levels of all the other gene modules at a time preceding the particular time, each of the coefficients representing an amount of effect that the expression level of one of the gene modules may have on the expression level of another one of the gene modules;
(C) selecting values for the coefficients that minimize a measure of the difference between a plurality of the measured expression levels and a plurality of the predicted expression levels.

8. A method according to claim 7, wherein a predicted value of the expression level of one of the gene modules at an initial time is calculated according to a function of the plurality of coefficients and measured values of the expression levels of all of the other gene modules.

9. A method according to claim 8, wherein all predicted values of the expression level of the one gene module at times following the initial time are calculated according to a function of the plurality of coefficients and predicted values of the expression levels of all the other gene modules.

10. A method according to claim 7, wherein selecting values for the coefficients comprises:
(A) assigning initial values to each of the coefficients;
(B) using the coefficients to calculate predicted expression profiles for at least some of the modules;
(C) selecting new values for the coefficients according to a function of a difference between the predicted expression profiles and the measured expression profiles.

11. A method according to claim 7, wherein selecting values for the coefficients comprises identifying two or more candidates for at least one of the coefficient values and setting the one coefficient value equal to an average of the candidates.
